(19) United States
(12) Patent Application Publication
(10) Pub. No.: US 2010/0324146 A1
(43) Pub. Date: Dec. 23, 2010

(54) Dendrimeric Enzyme Inhibitors

(75) Inventor: Jacinth Kincaid Fairley, Hawthorn (AU)

Correspondence Address: TECHNOLOGY LAW, PLLC, 3595 N. SUNSET WAY, SANFORD, MI, 48657, US

(73) Assignee: Starpharma Pty Ltd, Melbourne (AU)

(21) Appl. No.: 12/867,944

(22) PCT Filed: Feb. 20, 2009
(86) PCT No.: PCT/AU2009/000196

§ 371 Date: Aug. 17, 2010

(30) Foreign Application Priority Data
Feb. 22, 2008 (AU) 2008900859

(51) Int. Cl. A61K 31/166; C12N 9/99; A61P 17/02; A61P 19/02; A61P 9/10

(52) U.S. Cl. 514/618; 435/184; 514/620

(57) ABSTRACT

The present invention relates generally to the use of dendrimer compounds bearing anionic surface groups as inhibitors of hyaluronidase and their use in the treatment of diseases and conditions in which excessive, abnormal or unwanted levels of hyaluronidase are involved or implicated, or in the treatment of diseases or conditions in which the presence or administration of hyaluronic acid is therapeutically effective. The present invention also relates to pharmaceutical compositions and methods for the treatment of such diseases and conditions.

### FIELD OF THE INVENTION

The present invention relates generally to the use of dendrimer compounds bearing aryl acid, particularly anionic, surface groups as inhibitors of hyaluronidase and their use in the treatment of diseases and conditions in which excessive, abnormal or unwanted levels of hyaluronidase are involved or implicated, or in the treatment of diseases or conditions in which the presence or administration of hyaluronic acid is therapeutically effective. The present invention also relates to pharmaceutical compositions and methods for the treatment of such diseases and conditions.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Dendrimers are macromolecular highly branched compounds formed by reiterative reaction sequences starting from an initial, core molecule with layers or stages being added in successive “generations’ to build up a three-dimensional, highly ordered polymeric compound. Dendrimers may be generally characterised by the following features: (i) an initiator core (I) which may have one or more reactive sites and be point-like or of significant size so as to effect the final topology of the dendrimer; (ii) one or more layers of branched repeating units attached to the initiator core; (iii) functional terminal groups, such as anionic or cationic groups, attached, optionally through linking groups, to the surface of the dendrimer.

International Patent Application Nos. PCT/AU95/00350 (WO95/34595), PCT/AU99/00763 (WO 00/15240) and PCT/AU02/00407 (WO 02/00407), the contents of which are incorporated by reference, disclose dendrimers with a defined envelope of polyanionic or cationic surface groups, which have been shown to exhibit a range of antiviral and antimicrobial activity with minimal toxicity.

For example, polylysine, polyamidoamine (PAMAM), poly(etherhydroxylamine) (PEHAM) and polypropyleneimine dendrimers including, the compounds represented by Formulae (I) to (III) below, and bearing naphthyl disulphonate surface groups, have been shown to exhibit antimicrobial activity, particularly antiviral activity against a broad spectrum of pathogens associated with sexually transmitted diseases.

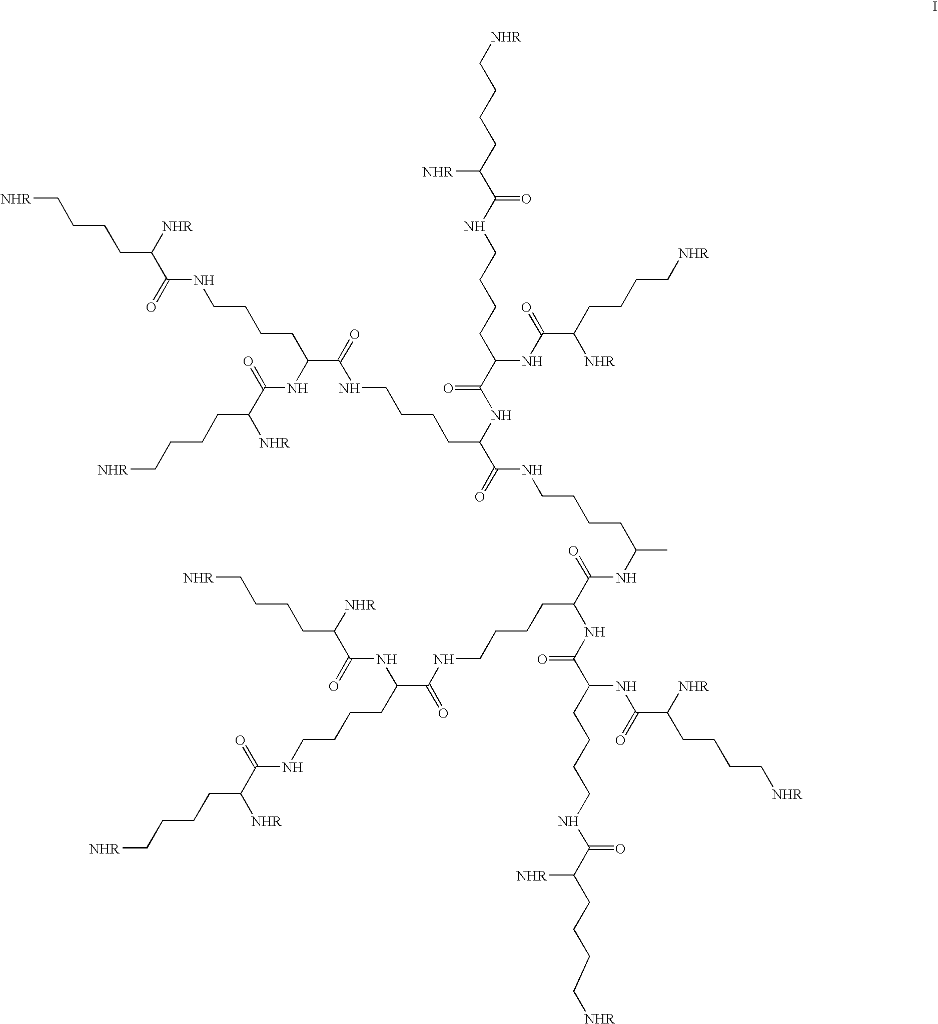

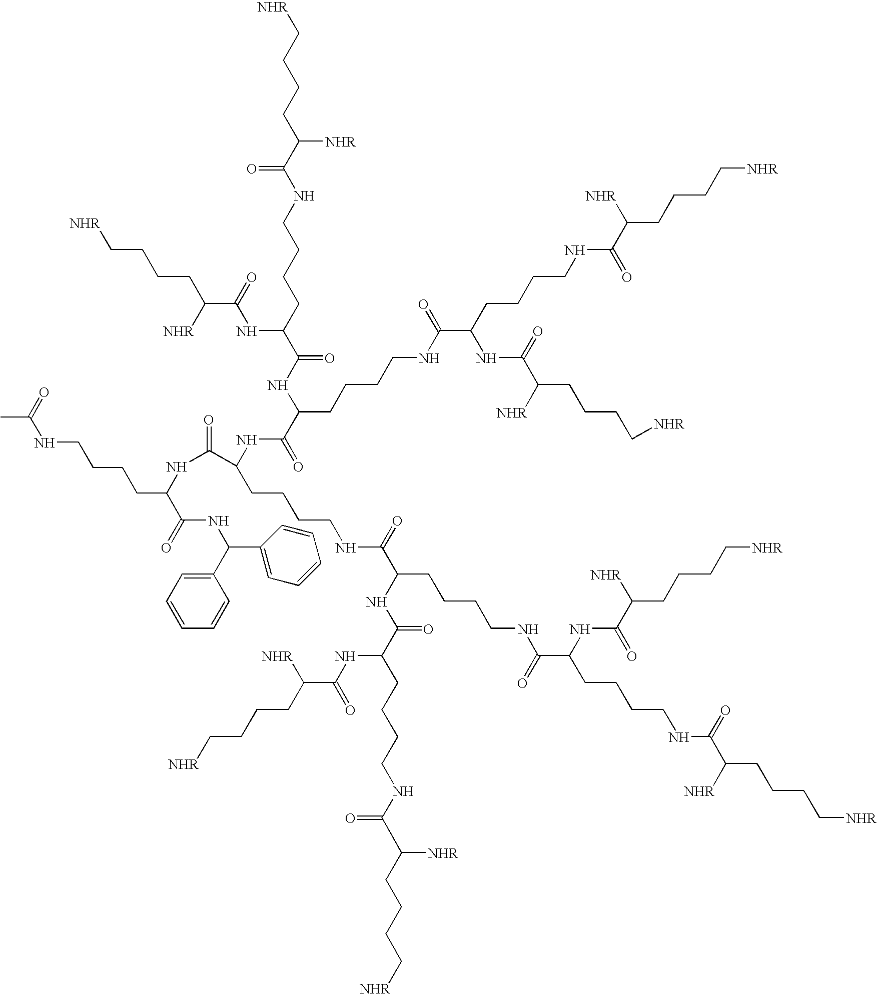

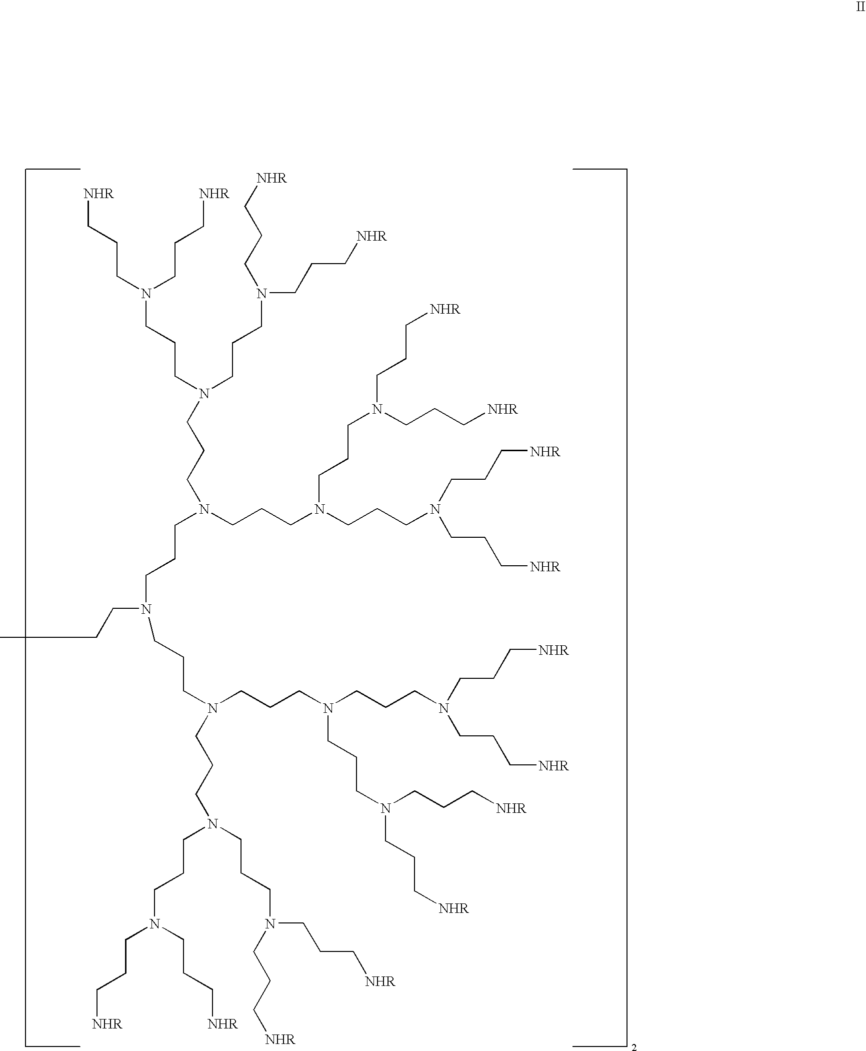

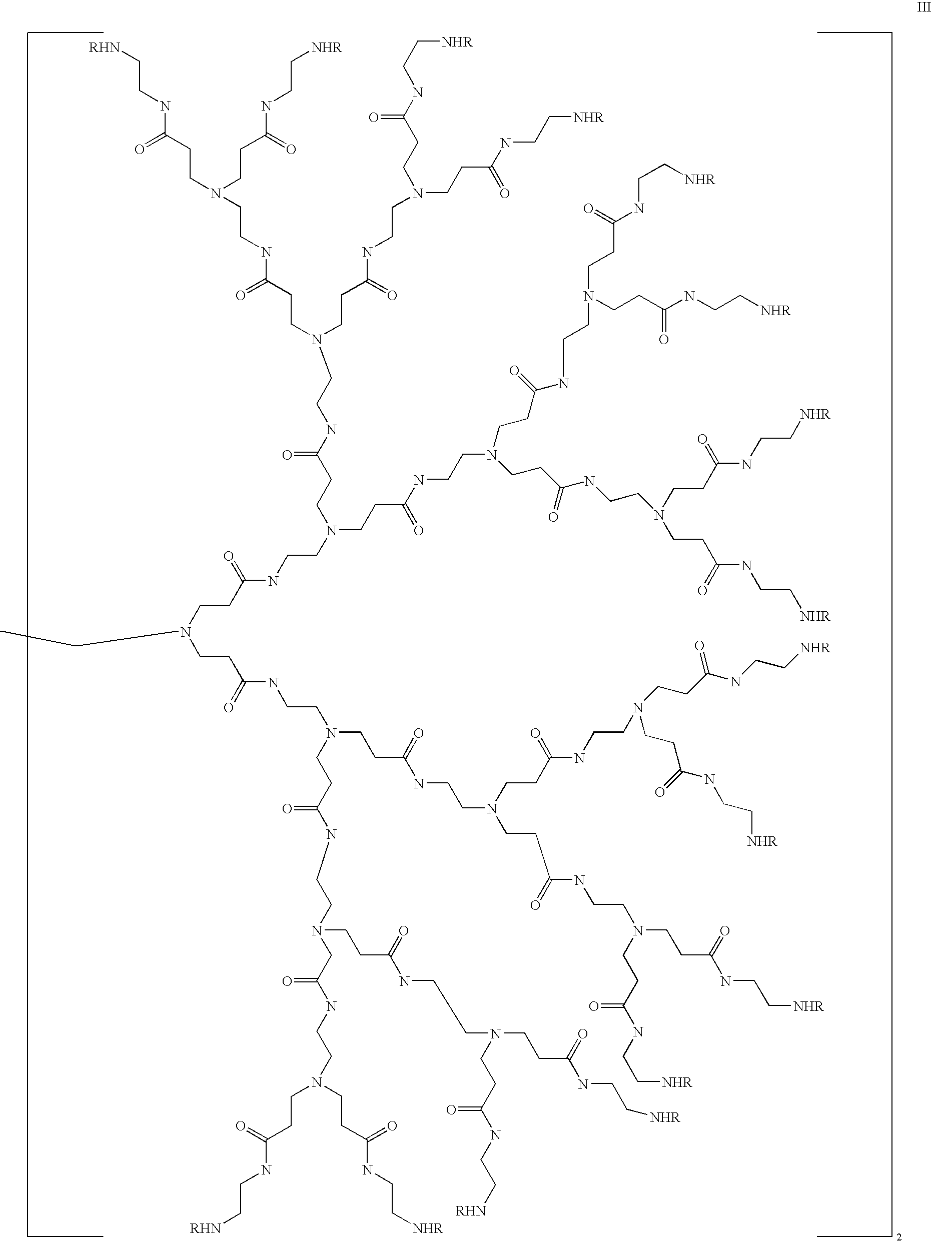

where R represents a group of the Formula (IV):

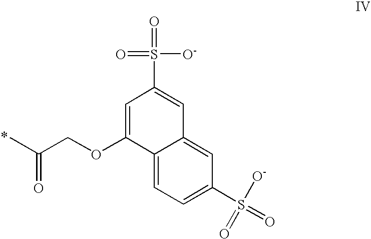

PCT/AU2007/000352 (WO 2007106944) further describes the contraceptive capacity of dendrimer compounds, such as those of Formulae (I)-(III), having surface anionic naphthyl disulfonic acid (otherwise referred to as naphthyl disulfonate) groups, as well as their use in contraceptive compositions.

Hyaluronan (also called hyaluronic acid or hyaluronate) is a non-sulfated glycosaminoglycan distributed widely throughout connective, epithelial, and neural tissues. It is composed of disaccharides, themselves composed of D-glucuronic acid and D-N-acetylglucosamine, linked together via alternating β-1,4-and β-1,3-glycosidic bonds. Hyaluronan can be 25,000 disaccharide repeat units in length and can range from 5,000 to 20,000,000 Da in vivo.

Hyaluronidase refers to the group of enzymes which catalyse the random hydrolysis of 1,4-linkages between N-acetyl-beta-D-glucosamine and D-glucuronate residues in hyaluronan. In humans there are at least six types of hyaluronidase-like enzymes including HYAL-1, HYAL-2, HYAL-3, HYAL-4 and PH-20. The degradation products of hyaluronan include the oligosaccharides and very low-molecular weight hyaluronan.

Arthritis is a major cause of chronic disability. The severity, location and progression of the disease varies from person to person, but typically the symptoms include stiffness, pain, and inflammation of the joints. Osteoarthritis occurs commonly in the knees, hands, hips or feet and is caused by “wear and tear” of the cartilage that cushions the bones. In contrast, rheumatoid arthritis, which commonly occurs in the hands, feet, shoulders and knees, is an autoimmune disease wherein the body's own immune system starts to attack its own joints. The synovial membrane, which encloses the joint becomes inflamed, synovial fluid is produced in excess, which causes the capsule around the joint to stretch and cause pain. The inflammation eventually damages the cartilage. Another common type of arthritis is traumatic arthritis, whereby an injury to the joint (e.g. hip fracture) leads to avascular necrosis, cutting off the blood supply to the joint and resulting in degradation of the surrounding cartilage. Many other diseases and conditions have arthritis as a symptom, for example, lupus, ankylosing spondylitis, Behcet's syndrome, and Sjögren's syndrome. Treatments may involve medication, lifestyle changes (e.g. weight loss) and, in severe cases, surgery to partially or fully replace joints.

High-molecular-weight hyaluronan (average molecular weight 6-7×10⁶ Da) is a major component of synovial joint fluids and increases the viscosity of the fluid. Along with lubricin, it is one of the fluid's main lubricating components. It physically acts as a viscous lubricant for slow joint movements, such as walking, and as an elastic shock absorber during rapid movements, such as running. Reductions in the concentration and average molecular weight of hyaluronan in knee synovial fluids from patients with osteoarthritis or rheumatoid arthritis have been widely reported. These reductions indicate hyaluronan's involvement in the pathogenesis of these joint disorders and are reflected in the pathological changes of hyaluronan metabolism.

Hyaluronidase-1, -2, and -3 are expressed in the synovium and are responsible for hyaluronan degradation (hydrolysis). Hyaluronan (Hylan GF-20) is used to treat osteoarthritis of the knee, shoulder and ankle. Such treatments, called viscosupplementation, are generally administered as a course of injections into the joint and are believed to supplement the viscosity of the joint fluid, thereby lubricating the joint, cushioning the joint, and producing an analgesic effect.

Hyaluronan is also an important component of articular cartilage, where it is present as a coat around each cell (chondrocyte). When aggrecan monomers bind to hyaluronan in the presence of link protein, large highly negatively-charged aggregates form. These aggregates imbibe water and are responsible for the resilience of cartilage (its resistance to compression). Clinical data suggests that elevated levels of hyaluronidase can contribute to cartilage degradation. The molecular weight (size) of hyaluronan in cartilage decreases with age, but the amount increases. It has also been suggested that hyaluronan has positive biochemical effects on cartilage cells. Oral use of hyaluronan has been lately suggested. At present, there are some preliminary clinical studies that suggest that oral administration of Hyaluronan has a positive effect on osteoarthritis.

Hyaluronan is also a major component of skin, where it is involved in tissue repair and wound healing and is found is high concentration in extracellular matrix whenever tissue repair occurs after injury and is laid down early in the matrix of both foetal and adult wounds. Current models of wound healing propose that larger polymers of hyaluronic acid appear in the early stages of healing to physically make room for white blood cells, which mediate the immune response. When skin is excessively exposed to UVB rays, it becomes inflamed (sunburn) and the cells in the dermis stop producing as much hyaluronan, and increase the rate of its degradation. Hyaluronan degradation products also accumulate in the skin after UV exposure. As hyaluronan is naturally found in many tissues of the body, such as skin, cartilage, and the vitreous humor, it is therefore well suited to biomedical applications targeting these tissues and may be used postoperatively to induce tissue healing. The first hyaluronan biomedical product, Healon, was developed in the 1970s and 1980s by Pharmacia, and is approved for use in eye surgery (i.e., corneal transplantation, cataract surgery, glaucoma surgery and surgery to repair retinal detachment). Other biomedical companies also produce brands of hyaluronan for ophthalmic surgery.

Subsequent to an atherothrombotic event, embolism, or hemorrhage in either cardiac or brain tissue, the resulting acute myocardial infarction or stroke is characterised by a rapid inflammatory response. A key marker of this response is the production of hyaluronan and its deposition at the wound site. Clinical data suggests that high molecular weight hyaluronan promotes cell quiescence and supports tissue integrity, whereas generation of hyaluronan breakdown products (via hyaluronidase activity) is a signal that an injury has occurred and subsequently, the initiation of an inflammatory response. The breakdown of hyaluronan by hyaluronidase typically results in scar-tissue formation—which can trigger or contribute to atherosclerotic plaque formation.

In 2003 the FDA approved hyaluronan injections (Restylane®) for filling soft tissue defects. The effects of the therapy are typically 4-8 months and require repeat treatments.

Given the demonstrated therapeutic benefits of hyaluronan and, in certain circumstances, the potentially undesirable effects of hyaluronan breakdown by hyaluronidase, inhibiting the activity of hyaluronidase, and thereby preventing or postponing, reducing or slowing down the extent of hyaluronan degradation, may provide therapeutically useful outcomes or be useful as an adjunctive therapy.

### SUMMARY OF THE INVENTION

The present invention is predicated on the discovery that a dendrimer having one or more aryl acid surface groups inhibits the hyaluronan hydrolysis activity of hyaluronidase. In certain aspects the present invention therefore provides compositions and methods for inhibiting undesirable hyaluronidase activity in a subject, particularly in order to inhibit hyaluronan degradation or hydrolysis. Thus, the administration of such a hyaluronidase inhibitor may have a therapeutic ameliorating or prophylactic effect for diseases and conditions in which undesirable hyaluronidase activity plays a role, and/or may be a useful adjunctive therapy for diseases and conditions where the administration of hyaluronan is beneficial or efficacious.

Accordingly, in a first aspect, the present invention provides a method for inhibiting hyaluronidase activity comprising contacting hyaluronidase with an effective amount of a dendrimer molecule comprising one or more aryl acid groups, or a pharmaceutically acceptable salt thereof.

The inhibition of hyaluronidase may be performed in vitro, for example in screening to determine the degree or level of inhibitory activity, or may be performed in vivo, such as in a mammalian subject, particularly, although not exclusively, a human subject.

In a further aspect, the present invention provides a method of inhibiting hyaluronan degradation by hyaluronidase, in a subject in need thereof, comprising the administration of an effective amount of a dendrimer molecule comprising one or more aryl acid groups, or a pharmaceutically acceptable salt thereof.

The dendrimer compounds may be used to inhibit the degradation of endogenous hyaluronan and/or to inhibit the degradation of administered exogenous hyaluronan.

In certain embodiments of the invention, one or more of HYAL-1, HYAL-2, HYAL-3, HYAL-4 or PH-20 is inhibited.

In further embodiments, one or more of HYAL-1, HYAL-2 or HYAL-3 is inhibited.

In a still further aspect, the invention provides a method for treating arthritis in a subject in need thereof, comprising the administration of an effective amount of a dendrimer molecule comprising one or more aryl acid groups, or a pharmaceutically acceptable salt thereof. Some exemplary types of arthritis contemplated herein include osteoarthritis and rheumatoid arthritis.

In yet another aspect, the invention provides a method of preventing or reducing the formation of post-myocardial infarct or stroke scar tissue or atherosclerotic plaque, in a subject in need thereof, comprising the administration of an effective amount of a dendrimer molecule comprising one or more aryl acid groups, or a pharmaceutically acceptable salt thereof.

In yet another aspect, there is provided a method for treating a wound or promoting wound healing in a subject in need thereof comprising the administration of an effective amount of a dendrimer molecule comprising one or more aryl acid groups, or a pharmaceutically acceptable salt thereof.

In certain embodiments of the invention, the dendrimer molecule comprising one or more aryl acid groups or a pharmaceutically acceptable salt thereof is administered in conjunction with, either separately or together, sequentially or contemporaneously, hyaluronan.

Advantageously, in one or more embodiments the administration of the dendrimer molecule comprising one or more aryl acid groups, or a pharmaceutically acceptable salt thereof, may enhance or extends the longevity or effectiveness of the hyaluronan treatment.

Accordingly, in still a further aspect there is provided a method of enhancing or extending the effects of hyaluronan, in a subject in need thereof, comprising administering a dendrimer molecule comprising one or more aryl acid groups, or a pharmaceutically acceptable salt thereof in conjunction with said hyaluronan. The hyaluronan may be endogenous or administered exogenous hyaluronan.

The dendrimer may be administered together with hyaluronan, either as a separate formulation, or as a composition formulation. Alternatively the dendrimer may be administered before or after the hyaluronan.

In yet a further aspect, the present invention provides a combination comprising hyaluronan and a dendrimer molecule comprising one or more aryl acid groups or a pharmaceutically acceptable salt thereof. The combination may be administered as separate components, either separately or contemporaneously, or may be administered as a single composition of the two components.

In still another aspect, there is provided a composition comprising hyaluronan and a dendrimer molecule comprising one or more aryl acid groups or a pharmaceutically acceptable salt thereof, together with a pharmaceutically acceptable carrier.

In still further aspect, the invention provides for the use of a dendrimer molecule comprising one or more aryl acid groups, or a pharmaceutically acceptable salt thereof in the manufacture of a medicament for inhibiting hyaluronan degradation by hyaluronidase or extending or enhancing the effects of hyaluronan, treating arthritis, preventing post-myocardial infarct or stroke, scarring, preventing atherosclerotic plaque formation, or in promoting wound healing.

The dendrimer may be any suitable dendrimer, for example a polylysine, PAMAM, PEHAM or polypropyleneimine based dendrimer.

In certain embodiments, the aryl acid group is the anionic naphthyl disulfonate group of formula (IV):

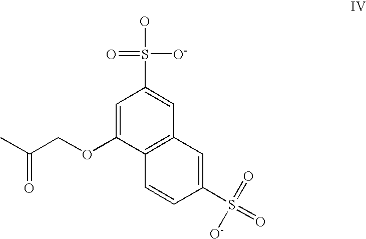

particularly in the form of a pharmaceutically acceptable salt, such as a disodium salt. In further embodiments of the invention, the dendrimer molecule is a compound of Formula (I), (II) or (III) as described herein. In one particular embodiment the dendrimer molecule is a compound of Formula (I).

Diseases and conditions contemplated as suitable for ameliorating or prophylactic treatment by the present invention include those hereinbefore described. Particular examples include rheumatoid arthritis, osteoarthritis, wounds and post-myocardial infarct or stroke scar formation, or atherosclerotic plaque formation.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The singular forms “a”, “an” and “the” include plural aspects unless the context clearly dictates otherwise.

The contents of the documents referred to herein are to be considered as incorporated into the present specification for all purposes by virtue of their reference.

As used herein, the term “inhibits”, or variations such as “inhibiting” or “inhibition”, includes the prevention, delay or postponement, slowing down or reduction in extent or rate of the ability of hyaluronidase to degrade or hydrolyse hyaluronan. This may be determined by detecting or measuring, either in vitro or in vivo, the presence of, or relative or absolute amounts of hyaluronan and/or its degradation (ie hydrolysis) products using methods known in the art. Some exemplary methods include those described in the Examples herein and by Nakamura et al, (1990); Krupa et al, (2003); Shimada et al, (1980); Takazono et al, (1984); Chun et al, (1988); Stern et al, (1992); Hautmann et al, (2004); and Wolf et al, (1984).

Unless specified, or the context indicates otherwise, reference to “hyaluronidase” refers to at least one of the subtypes of hyaluronidase. In certain embodiments of the invention, “hyaluronidase” refers to one or more of HYAL-1, HYAL-2, HYAL-3, HYAL-4 and PH-20.

Dendrimer compounds contemplated herein include any branched molecule comprising a core moiety to which is attached at least one layer or generation of building units or building blocks. In certain embodiments, the dendrimer contains at least 2, 3, 4, 5 or 6 layers or generations of building units.

The dendrimers may comprise a single type of building unit, or may comprises two or more different building units, for example, a single layer or generation may comprise two or more building units, or the dendrimer may comprise at least two layers or generations, each made up of a single, but different, type of building unit.

In further embodiments of dendrimers contemplated, each available position of a building unit which is not an outermost or surface building unit may be further coupled to a building unit. In other embodiments, one or more available positions on one or more building units may be blocked or protected to prevent further coupling with a building unit at that site and thereby form a dendrimer wherein the dendrimeric branching is terminated at that site. Suitable blocking or protecting groups are known in the art. In particular, suitable protecting groups for reactable amines include Boc, CBz, 4-nitrobenzyloxycarbamate (4-NO₂—CBz) Fmoc, Dde, CF₃CO₂, 2-halo-CBz, Alloc, Me₃SiEtSO₂, Troc, o-NO-₂PhSO₂ and 2,4-dinitrobenzene-sulfonyl groups.

In particular examples, of the invention, the building units contain active free amino groups for further coupling to the next generation of building units, blocking groups, linkers or to the surface naphthyl disulfonate groups.

Dendrimers contemplated herein may comprise lysine, or lysine analogue building units. The term “lysine analogue” refers to a molecule which has a single apex carboxyl group for attachment to the previous layer of building units, and two or three primary amine groups to which can be attached further building units, blocking groups, linkers or aryl acid groups. Examples of “lysine analogues” contemplated herein are described in PCT/AU2007/000352, for example glycyl-lys. In some particular examples, the dendrimer comprises only lysine or one type of lysine analogue as the building unit.

Other dendrimers contemplated herein include those comprising polyamidoamine (PAMAM), poly(etherhydroxylamine) (PEHAM) or polypropyleneimine building units. In particular examples thereof, the dendrimer has only polyamidoamine (PAMAM), poly(etherhydroxylamine) (PEHAM) or polypropyleneimine as the building unit.

The core moiety may contain only 1 point of attachment for a building unit or may contain 2, 3 or more points, which may or may not be further utilized for the attachment of building units. Typically, the point of attachment is a free amino group. Core moieties may consist of, comprise or be derived from a building unit or may be a molecule different to the building units. Exemplary core moieties are illustrated herein and described in PCT/AU2007/000352.

Methods for the preparation of dendrimers are well known in the art, see for example U.S. Pat. Nos. 4,289,872, 4,410,688, PCT/AU2006/001591 and PCT/AU2007/000352 (describing dendrimers based on layers of lysine or lysine analogue units), as well as U.S. Pat. Nos. 4,568,737 and 4,587,329 (describing dendrimers based on other units including polyamidoamine or PAMAM dendrimers). Dendrimers may be prepared in a convergent or divergent fashion.

Exemplary dendrimer scaffolds contemplated by the present invention include those depicted by Formulae (I), (II) and (III) as described herein.

As used herein, an “aryl acid group” refers to an aryl sulfonic acid, aryl phosphonic acid or aryl carboxylic acid group includes any group attached to the dendrimer surface and which contains an aryl sulfonic acid, phosphonic acid or carboxylic acid moiety. In certain embodiments, the aryl acid group advantageously includes a linker which attaches the aryl acid moiety to the dendrimer surface. Some exemplary linkers include: —C(═O)—CH₂O—*, —C(═S)—NH—*, —C(═O)—NH—*, —C(═O)—*, —(CH₂)₂—C(═O)—NH—* where * indicates the point of attachment to the aryl acid moiety. The aryl sulfonic acid, aryl phosphonic acid or aryl acid group may be used or administered in its protonated or anionic form, or pharmaceutically acceptable salt thereof. “Aryl” refers to phenyl or naphthyl and the aryl group may contain one, two or three acid groups. In further advantageous embodiments the aryl acid group is in anionic form, (sulfonate, phosphonate or carboxylate) particularly a pharmaceutically acceptable salt thereof. In some embodiments of the invention the anionic group is a phenyl or naphthyl mono-, di or trisulfonate group.

Exemplary aryl acid groups contemplated herein may include those specifically described in WO 00/15240 (PCT/AU99/00763), such as

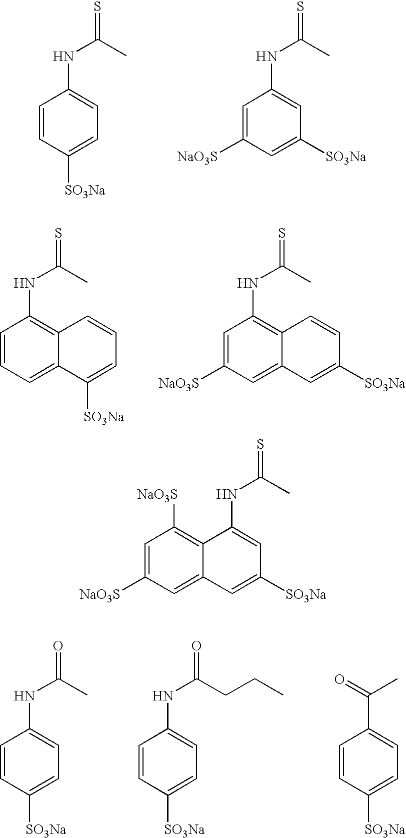

The term “surface” as used with reference to aryl acid groups, refers to the outermost layer of building units of the dendrimer, or of each dendritic motif as appropriate. A “dendritic motif” refers to a discrete unit of the molecule, for example, the branched structure arising from one or more successive layers or generations of building units attached to one point of the core moiety.

One or more aryl acid groups may be attached to the surface of the dendrimer or a dendritic motif. In certain embodiments, each available position on the surface of a dendrimer or dendritic motif bears an aryl acid group. In other embodiments the aryl acid groups are attached at defined available positions of the total dendrimer surface or dendritic motif surface, for example every second available position, eg at one free amino group, either the α or ε, amino groups available on lysine. Methods for controlling the placement or stoichiometry of the surface aryl acid groups are described in PCT/AU2006/001591.

The surface aryl acid groups may be attached directly to the outermost building blocks, or may be attached via any suitable linker group. Exemplary linker groups are described in PCT/AU2007/000352.

The aryl acid group may advantageously be in the form of a pharmaceutically acceptable salt. Suitable salts contemplated herein include those formed with pharmaceutically acceptable monovalent metal cations, such as sodium, potassium, lithium, or polyvalent cations, including divalent cations (e.g. magnesium, zinc and calcium) or trivalent cations (e.g. aluminium). An exemplary naphthyl disulfonate group contemplated herein is —COCH₂O-3,6-Naph(SO₃⁻)₂. An exemplary salt thereof is the disodium salt, eg —COCH₂O-3,6-Naph(SO₃Na)₂. Other salts include those formed with ammonium (NH₄⁺) or organic amine salts formed from, for example, alkyl (e.g. methyl, ethyl or propyl)amines N,N′-dibenxyl-ethylenediamine, chloroprocaine, diethanolamine, ethylenediamine, dicyclohexylamine, meglumine (N-methylglucamine), and procaine and quaternary amines such as choline, and sulphonium and phosphonium salts.

In particular embodiments of the invention, the dendrimer molecule contemplated is a compound of Formula (I), (II) or (III) wherein the surface group is the naphthyl disulfonate group of Formula (IV) or a pharmaceutically acceptable salt thereof. A further particular embodiment thereof is a compound of Formula (I), in particular the disodium salt thereof.

In certain embodiments, the dendrimer molecules contemplated herein are contemplated for use in therapeutic treatments. As used herein, “treatment”, in the appropriate context, may include ameliorating (eg of an existing disease/condition or symptom thereof) treatment or prophylactic (i.e. preventative) treatment and thus may include one or more of: preventing, alleviating, eliminating or reducing the frequency or severity of one or more symptoms, of or preventing or delaying the onset of, inhibiting the progression of, or halting or reversing (partially or altogether) the onset or progression of the particular disorder or condition being treated.

Subjects to be treated include mammalian subjects: humans, primates, livestock animals (including cows, horses, sheep, pigs and goats), companion animals (including dogs, cats, rabbits, guinea pigs), and captive wild animals (as found in zoos and wildlife parks). Laboratory animals such as rabbits, mice, rats, guinea pigs and hamsters are also contemplated as they may provide a convenient test system. Non-mammalian species such as birds, amphibians and fish may also be contemplated in certain embodiments of the invention.

An “effective amount” refers to an amount, when administered in accordance with a desired dosing regiment, fully or partially achieves the desired outcome. Thus a treatment effective amount is intended to include an amount which, when administered according to the desired dosing regimen, at least partially attains the desired therapeutic effect, including one or more of: alleviating, eliminating or reducing the frequency one or more symptoms, of, preventing or delaying the onset of, inhibiting the progression of, or halting or reversing (partially or altogether) the onset or progression of the particular disorder or condition being treated. An inhibiting effecting amount is an amount, when used or administered in accordance with a desired dosing regimen, at least partially inhibits the ability of hyaluronidase to degrade or hydrolyse hyaluronan.

The dendrimer molecules contemplated herein may be advantageously administered in conjunction with a therapeutic form of hyaluronan.

Thus, a further aspect of the invention provides a combination comprising hyaluronan and a dendrimer molecule comprising one or more naphthyl disulfonate groups, or a pharmaceutically acceptable salt thereof.

In one embodiment thereof the combination is in the form of a composition. The invention therefore further provides a composition comprising hyaluronan and a dendrimer molecule comprising one or more naphthyl disulfonate groups or a pharmaceutically acceptable salt thereof. Advantageously the composition further comprises a pharmaceutically active carrier.

Biofilms are structured communities of micro-organisms encapsulated in a polysaccharide matrix, adhering to a living or inert surface. They can form on any type of surface, including solid substrates, air-liquid interfaces and soft tissue, which is submerged or exposed to an aqueous medium. WO 00/15240 describes the anti-microbial properties of dendrimers such as that of Formulae (I)-(III). Accordingly, in certain embodiments of the invention, the compositions contemplated herein may possess the further advantage of inhibiting biofilm formation, thus potentially enhancing their shelf life.

The term “hyaluronan” (or “hyaluronic acid” or hyaluronate” or variants thereof) contemplates all forms thereof as appropriate, and includes native or endogenous forms, synthetic fauns, derivatized and non-derivatized forms, pharmaceutically acceptable salts and and crosslinked and non-cross-linked forms.

In certain embodiments of the invention, the hyaluronan is cross-linked, e.g. to form a water-insoluble gel or film. Advantageously, when hyaluronan is cross-linked in the presence of a dendrimer contemplated by the invention, the dendrimer may become entrapped within the cross-linked matrix. Methods for crosslinking hyaluronan may utilize polyfunctional aldehydes and epoxy compounds and polyhydric alcohols. All such suitable forms as appropriate are contemplated herein.

In further embodiments, the hyaluronan used in accordance with the present invention may be or derivatized hyaluronan. Derivatized forms of hyaluronan include modifications of one or more of the carboxyl groups such as O-esters (e.g. alkyl, benzyl) and N-acyl and O-acyl ureas etc.

Methods for the preparation of and forms of cross-linked and/or derivatized hyaluronan contemplated herein are known in the art and are described in, for example: Practical Aspects of Hyaluronan Based Medical Products, Jing-wen Kuo, CRC Press, (2005); WO 02/067994; WO 07/070547; The Chemistry, Biology and Medical Applications of Hyaluronan and its Derivatives (Laurent T C, ed) Wenner-Gren International Series, Vol 72, Portland Press, London (1998); Prestwich, G. D. et al., (1997); Luo, Y. et al., Chemical modification of hyaluronic acid. In Methods of Tissue Engineering; A. Atala and R. Lanza, Ed.; Academic Press: San Diego; Pouyani, T. et al., (1994); Chen, G. P. et al., (1997); Laurent, T. C. et al., (1964); Kuo, J.-w. et al., (1991); Luo, Y. et al., (2000); Larsen, N. E. et al., Hylan and hylan derivatives in drug delivery. In Cosmetic and Pharmaceutical Applications of Polymers; C. G. Gebelein, Ed.; Plenum Press: New York, 1991; pp 147-157; Yerushalmi, N. et ali, (1994); Asayama, S. et al., (1998); Hardingham, T. E. Solution properties of hyaluronan. In Chemistry and Biology of Hyaluronan (Eds. Garg, H. G. & Hales, C. A.) 1-19 (Elsevier, Amsterdam, 2004); and Hardingham, T. E. Properties of hyaluronan in aqueous solution. In: Hyaluronan Structure, Metabolism, Biological Activities and Therapeutic Applications (Eds. Balazs, E. A. & Hascall, V. C.) 67-78 (Matrix Biology Institute, New Jersey, 2005).

The hyaluronan contemplated for the various forms and embodiments of the invention may differ in MW, degree of crosslinking, derivatization, etc according to the intended application. The attending physician will be able to determine the most suitable form, dosage amount and frequency for each type of application.

The dendrimer molecules, either as a sole therapeutic agent, or in conjunction with another therapeutic agent, such as hyaluronan, can be used in the therapeutic treatment of diseases and conditions in which undesirable hyaluronidase activity is involved or in which hyaluronan therapy is efficacious or beneficial. Thus, the dendrimer molecules may be used in the treatment of arthritis, e.g. rheumatoid arthritis or osteoarthritis, to promote the healing of wounds, to prevent scar and atherosclerotic plaque formation after an atherothrombotic event, embolism or hemorrhage in either cardiac or brain tissue, following opthalmic surgery, or as a cosmetic treatment to improve the aesthetic appearance of the skin, for example facial skin such as in the treatment (amelioration or improvement in the appearance) of wrinkles, fine lines and furrows.

As noted above, hyaluronan plays an important role in maintaining healthy and functioning joints, with hyaluronan viscosupplementation an emerging treatment for arthritic conditions. Furthermore, as hyaluronidase is considered to play a role in cartilage degradation, the administration of a hyaluronidase inhibitor will assist with inhibiting the degradation or hydrolysis of endogenous or administered exogenous hyaluronan.

Thus, the dendrimer molecules contemplated herein are contemplated as having utility in the treatment of arthritis and/or its symptoms.

Arthritis, as used herein, refers to all types of arthritis, including osteoarthritis, rheumatoid arthritis, traumatic arthritis, gout, fibromyalgia and arthritic symptoms associated with other diseases and conditions as hereinbefore described. Exemplary joints include: knee, hip, ankle, wrist, elbow, shoulder, hand and feet joints and vertebrae. In particular embodiments, the dendrimer may be administered parenterally, i.e. via injection into the joint, alone or in combination with hyaluronan.

Dendrimer molecules contemplated by the invention for the treatment of arthritis can be demonstrated as follows:

Rheumatoid Arthritis Model.

Induction of proteoglycan arthritis in BALB/c mice. High density proteoglycan (aggrecan) isolated from pooled human cartilage samples and depleted of chondroitin sulfate and keratan sulfate can be used to induce rheumatoid arthritis. Female BALB/c mice (Charles River, Kingston, N.Y.) are injected intraperitoneally with the proteoglycan emulsified in Freund's adjuvant. The first overt symptoms (swelling and erythema of the paws) mark the day of onset of arthritis.

Treatment of arthritic mice. To minimise variance in the thickness of joints, acutely arthritic animals with similar degrees of joint swelling are selected for treatment. Mice are injected intraperitoneally daily with Compound from day 0 to day 5. The thickness of wrist and ankle joints are measured with a microcaliper in frontal and sagittal orientations. Blood samples are collected from anesthetized animals before the first injection (day 0) and 24 hours after the last injection (day 6).

Compound I-mediated reduction in symptoms in arthritic mice. Compound treatment of arthritic mice is expected to result in a dose and time-dependent decrease in thickness of wrist and ankle joints. Inflammatory markers are also expected to be reduced in blood samples proportional to Compound treatment.

In another embodiment, the dendrimer molecules contemplated herein may be used to accelerate, assist, or otherwise promote wound healing or tissue regeneration, or reduce the extent of, prevent or otherwise minimise scarring during the healing period or improved functional or cosmetic outcome such as improved strength or elasticity or reduced thickness, redress or induration of a wound.

As used herein, “wound healing” refers to the partial or full restoration of tissue continuity or integrity which is disrupted by a wound. “Treatment” of a wound relates to the inducement, initiation, promotion, stimulation, acceleration, augmentation, improvement, progression or other advancement of one or more stages of the wound healing process.

Wounds contemplated by the invention may be internal wounds, e.g. where the external structural integrity of the skin is maintained, or external wounds, particularly cutaneous wounds, e.g. a dermal or epidermal wound. Examples of wounds contemplated include cuts and lacerations, surgical incisions or wounds including ophthalmic surgery, punctures, grazes, scratches, compression wounds, abrasions, friction wounds (e.g. nappy rash, friction blisters), decubitus ulcers (e.g. pressure or bed sores); thermal effect wounds (burns from cold and heat sources, either directly or through conduction, convection, or radiation, and electrical sources), UV exposure (e.g. sunburn), chemical wounds (e.g. acid or alkali burns) or wounds arising from pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, stomach or intestinal ulcers, oral wounds, including ulcers of the mouth, damaged cartilage or bone, amputation wounds and corneal lesions.

The present invention contemplates the use of dendrimer molecules as described herein in all applications where inhibition of hyaluronidase activity is desirable or efficacious, provided that the use is not for the purpose of preventing fertilization, i.e. as a contraceptive agent.

Compositions for wound healing comprising the dendrimer molecules, are in some embodiments, in the form suitable for topical administration, may advantageously contain one or more other agents which may assist or promote the wound healing process, including growth factors, local anaesthetics, analgesics, anti-fungal, anti-bacterial or anti-viral agents. Suitable compositions for wound healing include emulsions, pastes, ointments, lotions, creams, powders, gels, hydrogels, hydrocolloids, aerosols and foams. Alternatively, the dendrimer molecules may be in the form of an occlusive dressing, i.e. where the compound is impregnated or coated on a dressing such as a patch, bandage, gauze, tape, net, membrane, film or adhesive plaster.

Suitable dosage amounts and dosing regimens for the dendrimer molecules can be determined by the attending physician and may depend on the particular disease or condition being treated, the severity of the condition as well as the general age, health and weight of the subject. Suitable dosage amounts of dendrimer molecule may lie in the range of from 1 μg to 1 g per kg of body weight of compound, salt, or solvate, for example, 1 μg-1 mg, 1 mg-10 mg, 10-mg-50-mg, 50 mg-100 mg or 100 mg-500 mg. Dosages may be administered once, or multiple times daily, or one or more times weekly, fortnightly or monthly.

The dendrimer molecules contemplated herein may be administered as the sole active therapeutic agent or may be administered in association with one or more other therapeutic agents. In particular embodiments, the dendrimer molecules contemplated herein may be administered in conjuction with hyaluonan therapy as determined by the administering physician. Thus, the dendrimer molecules contemplated herein may be administered contemporaneously with another agent, such as hyaluronan, either as separate compositions or as a single composition. Alternatively, the administration of the dendrimer molecule and one or more additional agents, such as hyaluronan, may be separated by an interval of time, such as 1-5 minutes, 10-30 minutes, 1-4 hours, 12-24 hours or other interval as deemed appropriate by the attending physician, whereby the dendrimer molecule can be administered before or after the other therapeutic agent, or in between doses of such an agents administration regimen.

In certain embodiments of the invention, the use of a dendrimer moleculecontemplated herein may, by inhibiting hyaluronidase activity, avoid or delay the need for additional treatment, or may enhance or prolong the beneficial effect of hyaluronan therapy such that reduced dosage amounts or increased intervals between hyaluronan dosages is permitted whilst retaining a desirable level of efficacy.

The active ingredient may be administered in a single dose or a series of doses. While it is possible for the active ingredient to be administered alone, it is preferable to present it as a composition, preferably as a pharmaceutical composition, with one or more pharmaceutically acceptable carrier excipients or additives. Thus, the present invention also relates to the use of a dendrimer molecule as described herein or a pharmaceutically acceptable salt thereof in the manufacture of a medicament for treatment of a disease or condition in which undesirable hyaluronidase activity is involved or in which hyaluronan therapy is efficacious or beneficial.

The formulation of such compositions is well known to those skilled in the art, see for example, Remington's Pharmaceutical Sciences, 18^{th} Edition, Mack Publishing, 1990. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, nasal, topical (including dermal, buccal and sublingual), vaginal or parental (including subcutaneous, intramuscular, intra-articular, intraocular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent), preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Devices for transdermal delivery, such as patches, may also be used to administer the compounds of the invention.

Compositions for rectal administration may be presented as ointments, lotions, creams, gels, pastes, foams or spray formulations or a suppository with a suitable base comprising, for example, cocoa butter, glycerin, gelatin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described,

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

If appropriate, the dendrimer molecules may also be administered as solvates. The term “solvate” refers to a complex or aggregate formed by one or more molecules of a solute, ie dendrimers contemplated by the invention, and one or more molecules of a solvent. Suitable solvents are well understood in the art and include for example, of water, ie to form hydrates, and common organic solvents such as alcohols (methanol, ethanol, isopropanol) and acetic acid. Methods of solvation are generally known within the art, for example, recrystallization from an appropriate solvent.

The invention will now be described with reference to the following examples which are intended to illustrate certain aspects and/or embodiments of the invention and are not intended to limit the generality hereinbefore described.

### EXAMPLES

### Example 1

Compound I (represented hereinbefore by Formula (I) where R is the disodium salt of Formula (IV)) was tested for hyaluronidase inhibition activity.

Hyaluronidase Inhibition

Hyaluronidase activity was quantified by measuring the extent of hyaluronic acid hydrolysis. This was measured by determining the concentration of N-acetylglucosamine-like material, resulting from enzyme action.

Reaction mixtures contained the following, in a total volume of 0.25 mL: 0.1 M sodium acetate, containing 0.15 M NaCl, pH 5.5 (Acetate buffer); 7.2 Units sheep testicular hyaluronidase (Sigma Chemical Company, St. Louis, Mo.; Type III, cat. no. H-2251), from a stock solution dissolved in the Acetate buffer; 0.3 mg/mL hyaluronic acid (Sigma; from bovine vitreous humor, cat. no. 11-7630).

In this test and all tests presented herein, a 5-10× concentrated solution of Compound I was freshly prepared in the appropriate assay buffer just prior to use. Compound I was initially screened at a concentration of 1 mg/mL. Enzyme was pre-incubated with Compound I for 10 minutes prior to starting the reaction by adding hyaluronic acid. Enzyme activity was determined by the method of Aronson and Davidson (J Biol Chem 242: 437-440, 1967). Incubations were carried out for 30 minutes at ambient temperature. Reaction product was measured colorimetrically with a Beckman DU-7500 spectrophotometer after reaction with p-dimethylaminobenzaldehyde (Reissig et al., J Biol Chem 217: 959-966, 1955)).

Immediately after adding colorimetric reagent, Compound I in the reactions produced a precipitate, which was removed by filtration through a 0.45 μm syringe filter (Whatman Puradisc 25 AS; cat no. 6780-2504). Color was developed by incubation at 37 C for 30 min, and the absorbency of the resultant adduct was immediately determined at 545 nm. Absorbencies of blanks and errors of measurement in this experiment was relatively high, likely due to continued precipitation of Compound I.

The procedure was further modified, such that the reactions were filtered immediately prior to determining the absorbency at 545 nm. Variability of the dose-response measurements was considered acceptable (average coefficient of variance over all concentrations=6%).

Differences were determined between absorbencies for reactions run in the presence and absence of hyaluronidase (blank, in which hyaluronidase is added after terminating the reaction). For the dose-response and reversibility experiments, data are reported as absorbencies at 545 nm.

A dose-response curve was generated at several concentrations (6) of Compound I, ranging from 1 μg/mL to 500 μg/mL. The IC50 value (that concentration of inhibitor that produces 50% inhibition under the conditions of the assay), as well as the concentration of Compound I that would yield a 3-Log reduction in activity (99.9% inhibition), were determined by analyzing enzyme activity as a function of inhibitor concentration, with curve-fitting software (TableCurve 2D, version 5.0; SPSS Statistical Software, Chicago, Ill.).

Reversibility of hyaluronidase inhibition was determined by the method of Ackermann and Potter (Proc Soc Exp Biol Med 72: 1-9, 1949), in which the level of inhibited enzyme activity at a fixed concentration of Compound I is determined in the presence of different amounts of enzyme. A plot (enzyme activity in the presence of inhibitor vs. amount of enzyme) that passes through the origin suggests reversible inhibition. A Y-intercept below the origin suggests irreversible inhibition. Inhibition is considered kinetically irreversible if the Y-intercept of the inhibited reaction is significantly below that of the control reaction (i.e., confidence limits of the intercepts for the two groups do not overlap).

Results:

Compound I caused precipitation in hyaluronidase assay mixtures during chromophore development, thus interfering with spectrophotometric readings. This problem was resolved by filtering all reactions with 0.45 μm syringe filters just before spectrophotometric readings, as described in Methods.

Compound I inhibits hyaluronidase in a dose-dependent manner at concentrations ranging from 1.0 to 500 μg/mL. The IC50 value and concentration of Compound I required to inhibit hyaluronidase by 3-Logs (99.9%) were calculated as 48 μg/mL and 0.78 mg/mL, respectively. (See Table 1) An Ackermann and Potter plot of hyaluronidase activity as a function of enzyme with and without 0.05 mg/mL Compound I suggests that hyaluronidase inhibition by Compound I is reversible. Linear curves fit to the data have Y-intercepts whose 90% confidence limits overlap.

TABLE 1
Determination of Dose-Response for Hyaluronidase Inhibition

| [Com- | Absorbance at 545 nm | | | | | |
|---|---|---|---|---|---|---|
| pound 1] | | Reaction | | | Aver, | |
| (μg/mL) | Blank* | 1 | 2 | 3 | less Blank | SEM |
| 0 | 0.0013 | 0.1674 | 0.1579 | 0.1774 | 0.166 | 0.0056 |
| 1 | 0.0013 | 0.1682 | 0.1752 | 0.1596 | 0.166 | 0.0045 |
| 5 | 0.0013 | 0.1585 | 0.1611 | 0.1684 | 0.161 | 0.0030 |
| 10 | 0.0013 | 0.1366 | 0.1472 | 0.1536 | 0.145 | 0.0050 |
| 50 | 0.0013 | 0.0792 | 0.0865 | 0.0901 | 0.084 | 0.0032 |
| 100 | 0.0013 | 0.0432 | 0.0493 | 0.0583 | 0.049 | 0.0044 |
| 500 | 0.0013 | 0.0076 | 0.0079 | 0.0077 | 0.006 | 0.0001 |

TableCurve Equation y = a + b(1 − exp(−((x + dln(2)^{l/e} − c)/d)^{e})) Weibull Cumulative)
Coefficient of Determination (r²) 0.99999
IC₂₀ 14.9 μg/mL
IC₅₀ 48.4 μg/mL
IC₈₀ 0.13 mg/mL
3-Log reduction 0.78 mg/mL
*Blanks received hyaluronidase after adding borate buffer and NaOH to the tubes (after the 30 minute incubation).

### BIBLIOGRAPHY

Ackermann and Potter, Proc Soc Exp Biol Med 72: 1-9, 1949
Asayama, S., Nogawa, M., Takei, Y., Akaike, T., and Maruyama, A. (1998) Synthesis of novel polyampholyte comb-type copolymers consisting of a poly(L-lysine) backbone and hyaluronic acid side chains for a DNA carrier. Bioconjugate Chem. 9, 476-481
Chen, G. P., Ito, Y., Imanishi, Y., Magnani, A, Lamponi, S., and Barbucci, R. (1997) Photoimmobilization of sulfated hyaluronic acid for antithrombogenicity. Bioconjugate Chem. 8, 730-734
Chun L E, Koob T J, Eyre D R., Anal Biochem. 1988 May 15; 171(1):197-206
Hardingham, T. E. Solution properties of hyaluronan. In Chemistry and Biology of Hyaluronan (Eds. Garg, H. G. & Hales, C. A.) 1-19 (Elsevier, Amsterdam, 2004)
Hardingham, T. E. Properties of hyaluronan in aqueous solution. In: Hyaluronan Structure, Metabolism, Biological Activities and Therapeutic Applications (Eds. Balazs, E. A. & Hascall, V. C.) 67-78 (Matrix Biology Institute, New Jersey, 2005)
Hautmann S, Toma M, Lorenzo Gomez M F, Friedrich M G, Jackel T, Michl U, Schroeder G L, Huland H, Juenemann K P, Lokeshwar V B., Eur Urol. 2004 October; 46(4):466-71 J Biol Chem 242: 437-440, 1967
Krupa J C, Marie Butler A, Mort J S., Anal Biochem. 2003 Aug. 15; 319(2):280-6
Kuo, J.-w., Swann, D. A., and Prestwich, G. D. (1991) Chemical modification of hyaluronic acid by carbodiimides. Bioconjugate Chem. 2, 232-241
Kuo J-w., Practical Aspects of Hyaluronan Based Medical Products, CRC Press, 2005
Larsen, N. E., Leshchiner, E. A., Parent, E. G., and Balazs, E. A. Hylan and hylan derivatives in drug delivery. In Cosmetic and Pharmaceutical Applications of Polymers; C. G. Gebelein, Ed.; Plenum Press: New York, 1991; pp 147-157
Laurent, T. C., Hellsing, K., and Gelotte, B. (1964) Cross-linked gels of hyaluronic acid. Acta Chem. Scand. 18, 274-275
Luo, Y., Kirker, K., and Prestwich, G. Chemical modification of hyaluronic acid. In Methods of Tissue Engineering; A. Atala and R. Lanza, Ed.; Academic Press: San Diego
Luo, Y., Kirker, K., and Prestwich, G. (2000) Cross-linked hyaluronic acid hydrogel films: new biomaterials for drug delivery. J. Controlled Release 69, 169-184
Nakamura T, Majima M, Kubo K, Takagaki K, Tamura S, Endo M., Anal Biochem. 1990 Nov. 15; 191(1):21-4
Pouyani, T., Harbison, G. S., and Prestwich, G. D. (1994) Novel hydrogels of hyaluronic acid: Synthesis, surface morphology, and solid-state NMR. J. Am. Chem. Soc. 116, 7515-7522
Prestwich, G. D., Marecak, D. M., Marecek, J. F., Vercruysse, K. P., and Ziebell, M. R. (1997) Controlled chemical modification of hyaluronic acid: synthesis, applications and biodegradation of hydrazide derivatives. J. Controlled Release 53, 99
Reissig et al., J Biol Chem 217: 959-966, 1955
Shimada E, Matsumura G., J Biochem. 1980 October; 88(4):1015-23
Stern M, Stern R., Matrix. 1992 November; 12(5):397-403
Takazono I, Tanaka Y., J Chromatogr. 1984 Apr. 20; 288(1):167-76
The Chemistry, Biology and Medical Applications of Hyaluronan and its Derivatives (Laurent T C, ed) Wenner-Gren International Series, Vol 72, Portland Press, London (1998)
Wolf R A, Glogar D, Chaung L Y, Garrett P E, Ertl G, Tumas J, Braunwald E, Kloner R A, Feldstein M L, Muller J E., Am J Cardiol. 1984 Mar. 15; 53(7):941-4
Yerushalmi, N., Arad, A., and Margalit, R. (1994) Molecular and cellular studies of hyaluronic acid-modified liposomes as bioadhesive carriers for topical drug delivery in wound healing. Arch. Biochem. Biophys. 313, 267-273;

1. A method for inhibiting hyaluronidase activity comprising contacting hyaluronidase with an effective amount of a dendrimer molecule comprising one or more aryl acid groups.

2. A method of inhibiting hyaluronan degradation by hyaluronidase, in a subject in need thereof, comprising the administration of an effective amount of a dendrimer molecule comprising one or more aryl acid groups.

3. The method according to claim 1 or 2 for treating arthritis.

4. The method according to claim 1 or 2 for preventing or reducing the formation of post-myocardial infarct or stroke scar tissue or atherosclerotic plaque.

5. The method according to claim 1 or 2 for treating a wound or promoting wound healing.

6. The method of enhancing or extending the effects of hyaluronan, in a subject in need thereof, comprising administering a dendrimer molecule comprising one or more aryl acid groups, in conjunction with said hyaluronan.

7. The method according to any one of claims 1, 2 or 6 wherein the aryl acid group is in anionic form or a pharmaceutically acceptable salt thereof.

8. The method according to claim 7 wherein the aryl acid group is a naphthyl disulfonate group.

9. The method according to any one of claims 1, 2 or 6 wherein the dendrimer molecule is of Formula (I), (II) or (III): 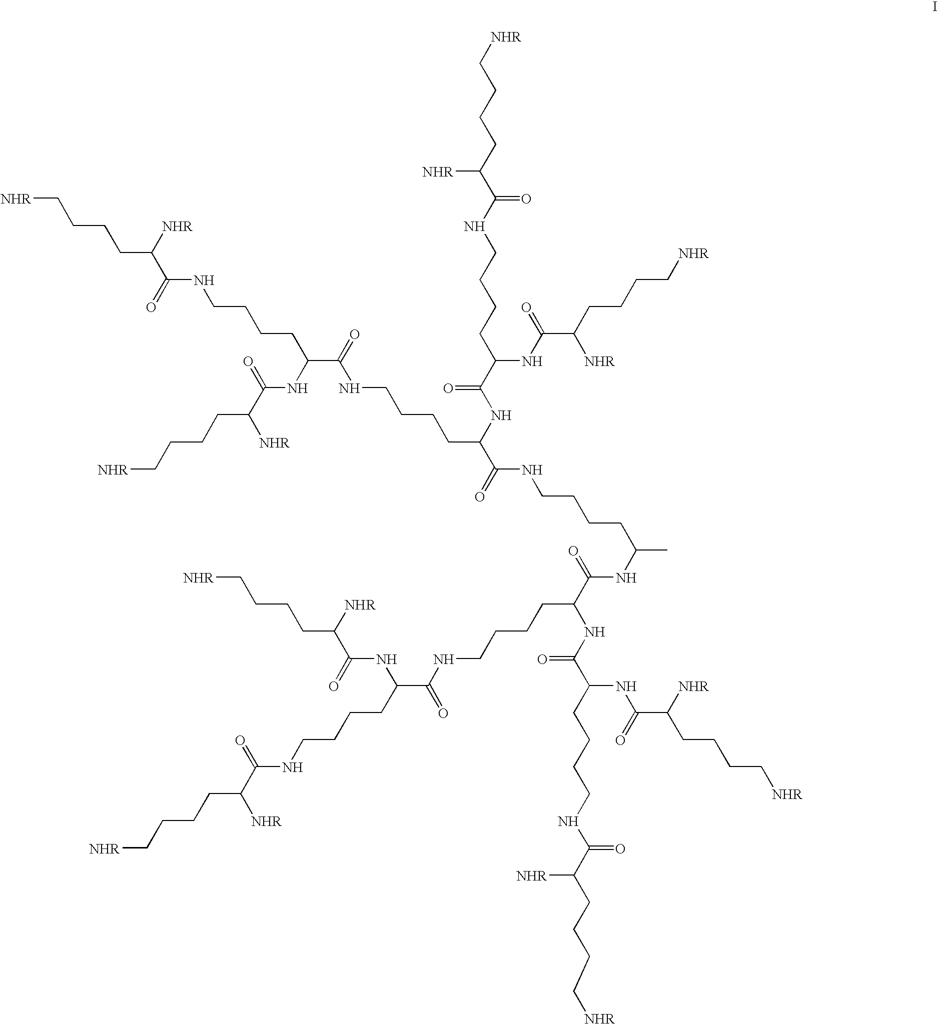 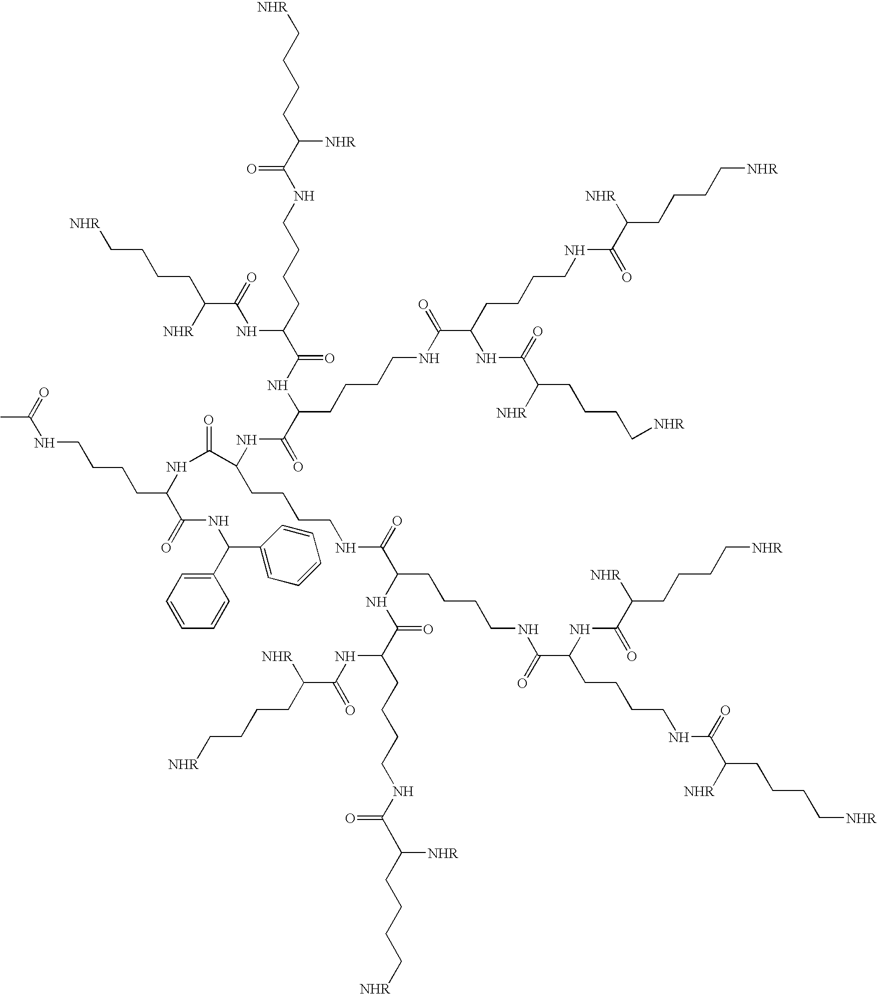 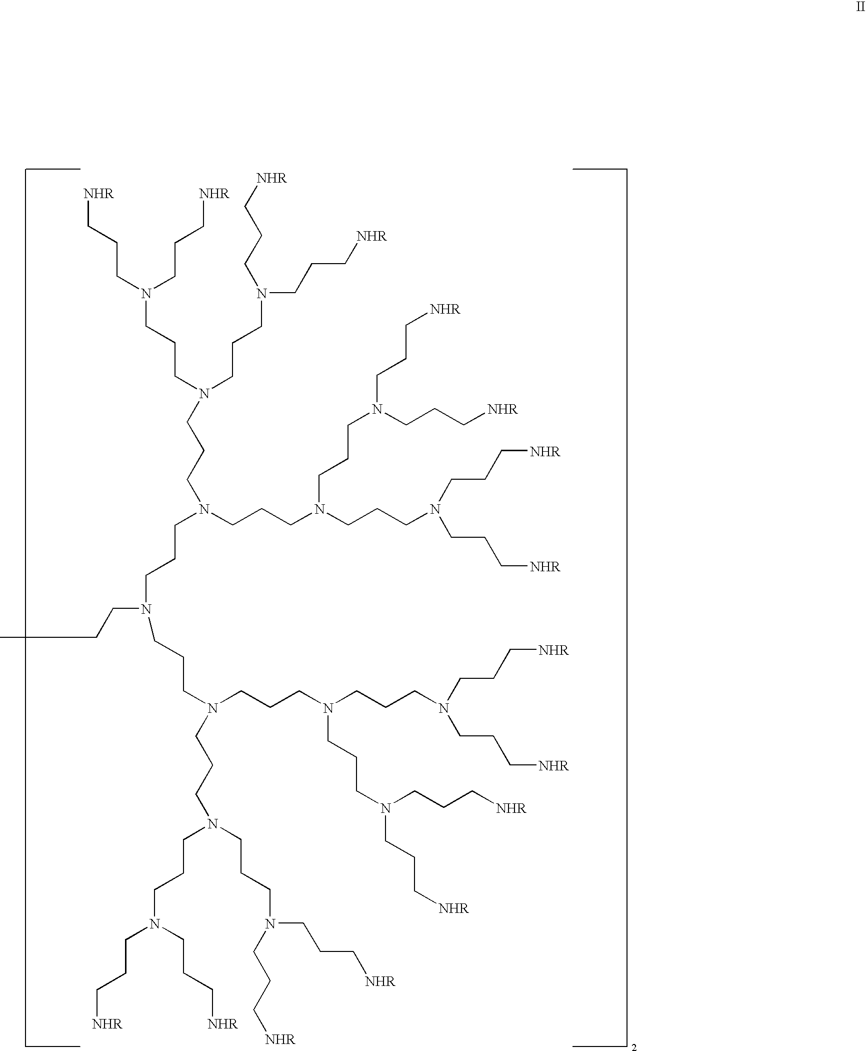 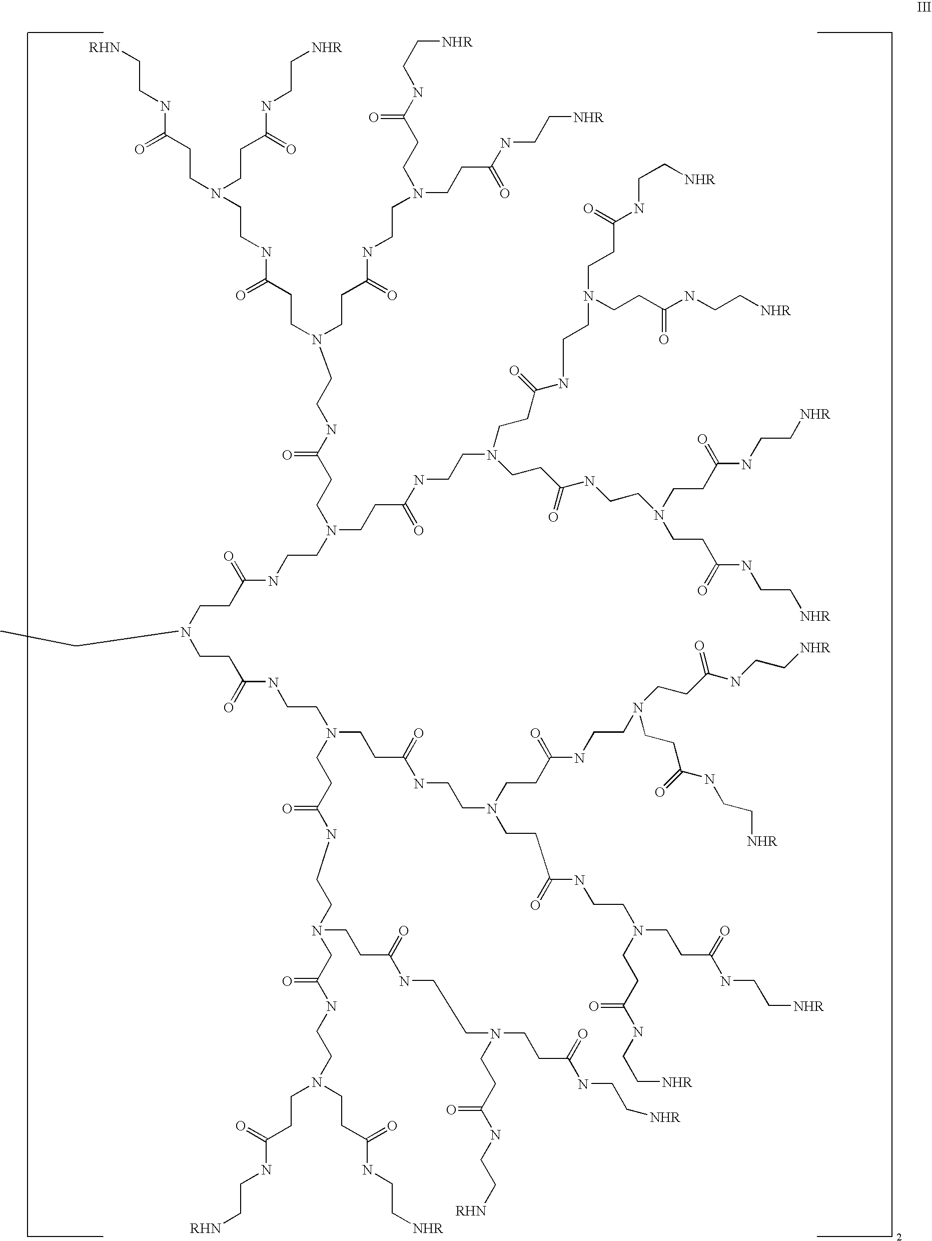
where R represents a group of the Formula (IV): 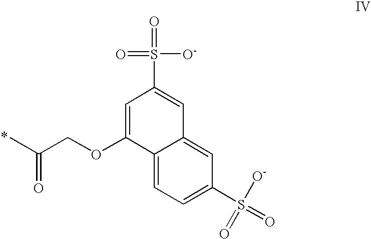
or a pharmaceutically acceptable salt thereof.

10. The method according to claim 9 wherein the dendrimer molecule is the disodium salt of Formula (I).

11. A combination comprising hyaluronan and a dendrimer molecule comprising one or more aryl acid groups or a pharmaceutically acceptable salt thereof.

12. The combination according to claim 11 wherein the dendrimer molecule is a compound of Formula (I), (II) or (III); 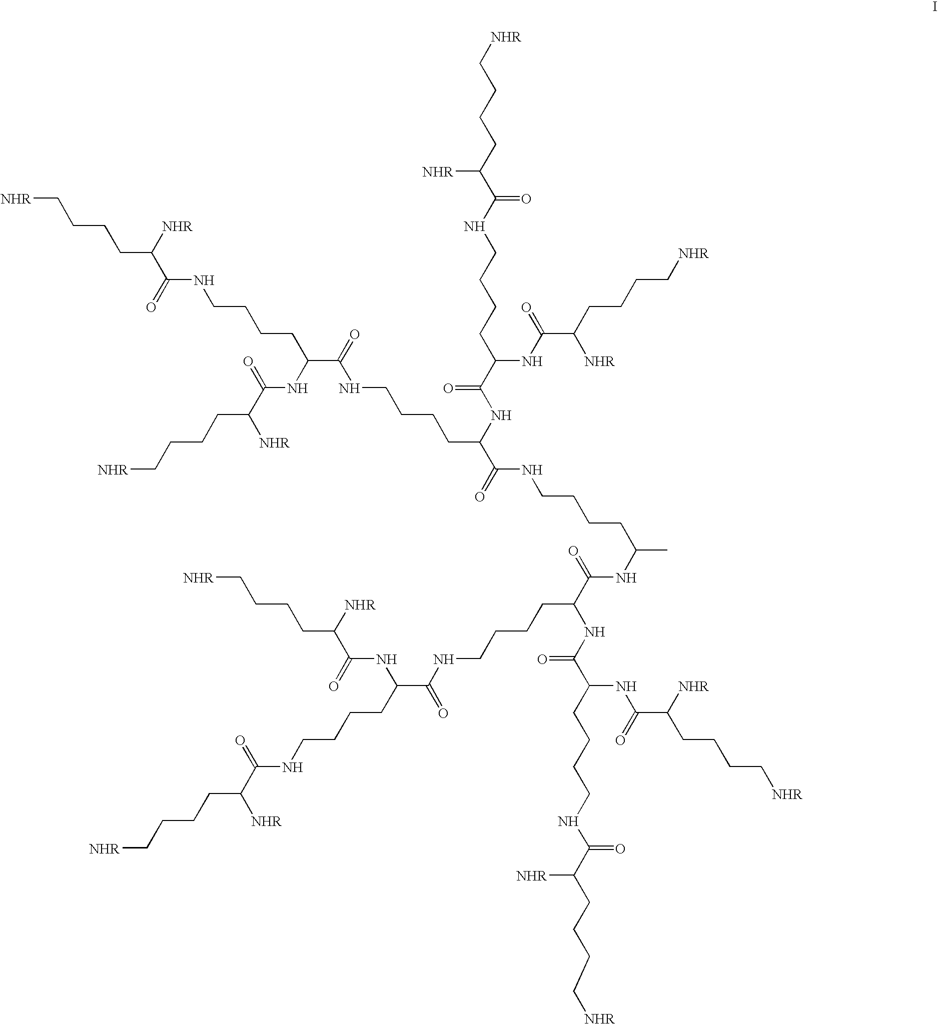 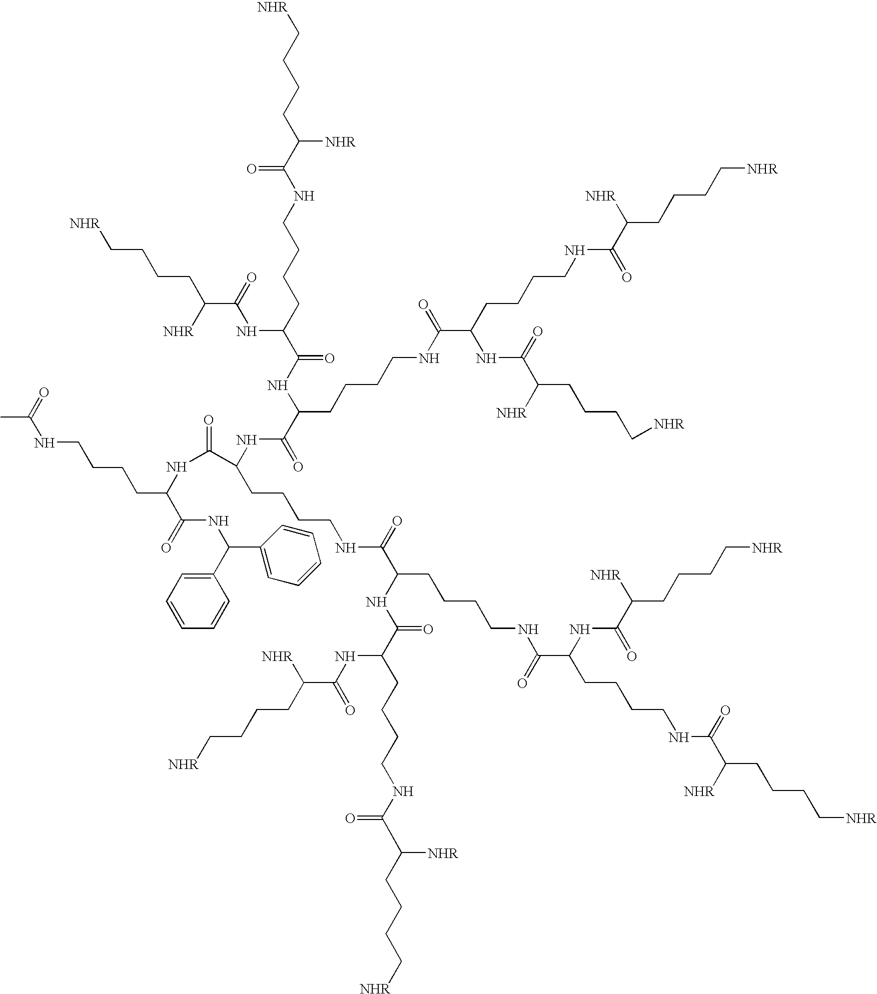 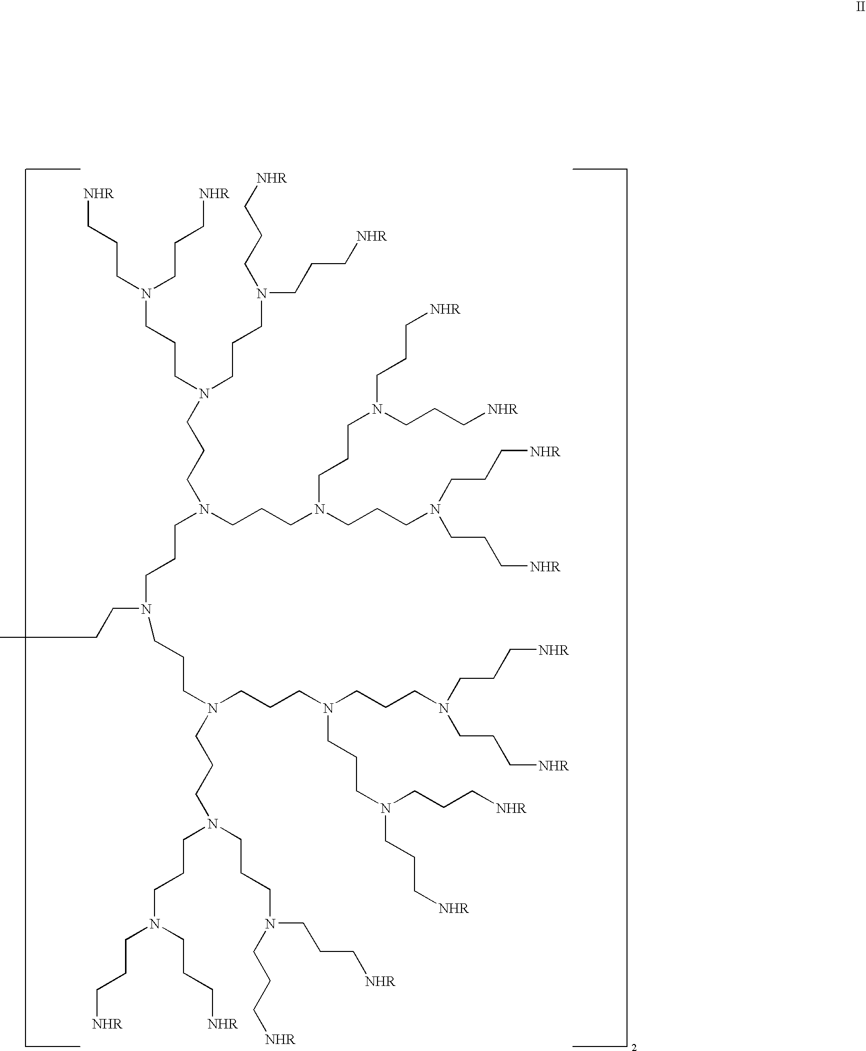 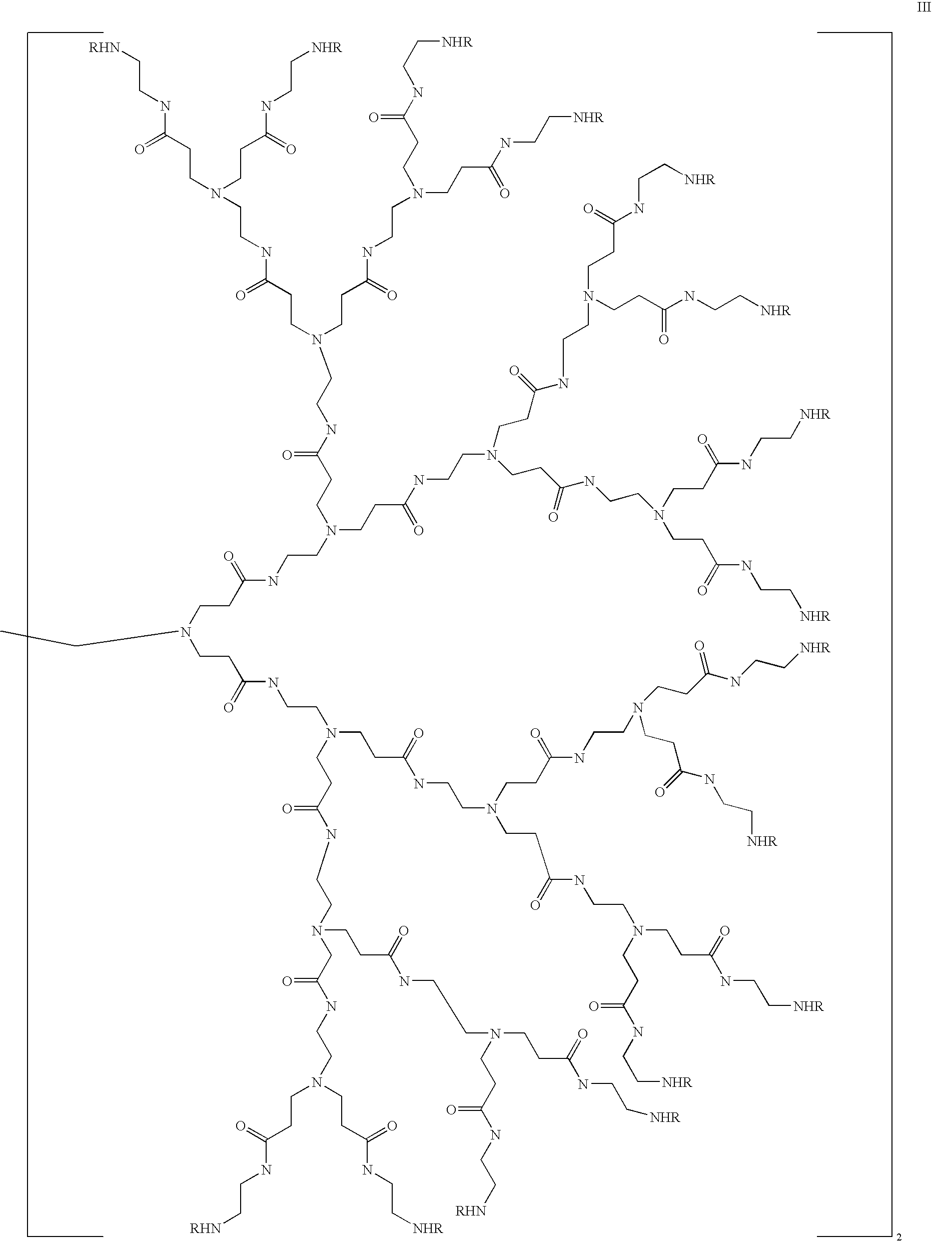
where R represents a group of the Formula (IV): 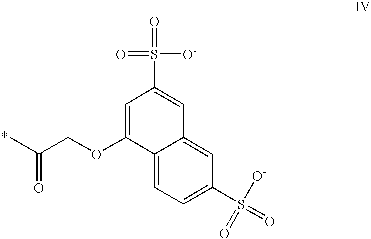
or a pharmaceutically acceptable salt thereof.

13. The combination according to claim 12 wherein the dendrimer molecule is the disodium salt of Formula (I).

14. (canceled)

15. (canceled)

16. The method of claim 1 or 2 wherein hyaluronan is administered sequentially or contemporaneously with the dendrimer molecule.

17. A composition comprising hyaluronan and a dendrimer molecule comprising one or more aryl acid groups or a pharmaceutically acceptable salt thereof.

18. The composition according to claim 17 wherein the dendrimer molecule is a compound of Formula (I), (II) or (III); 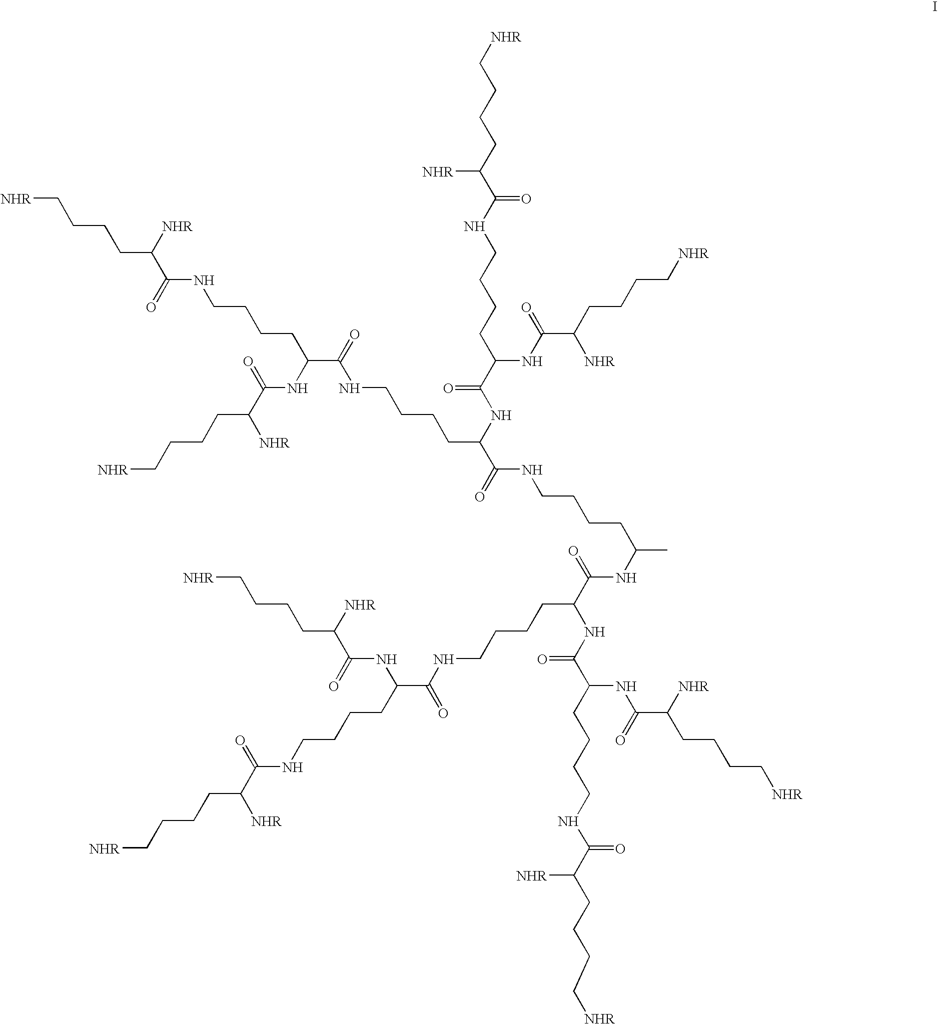 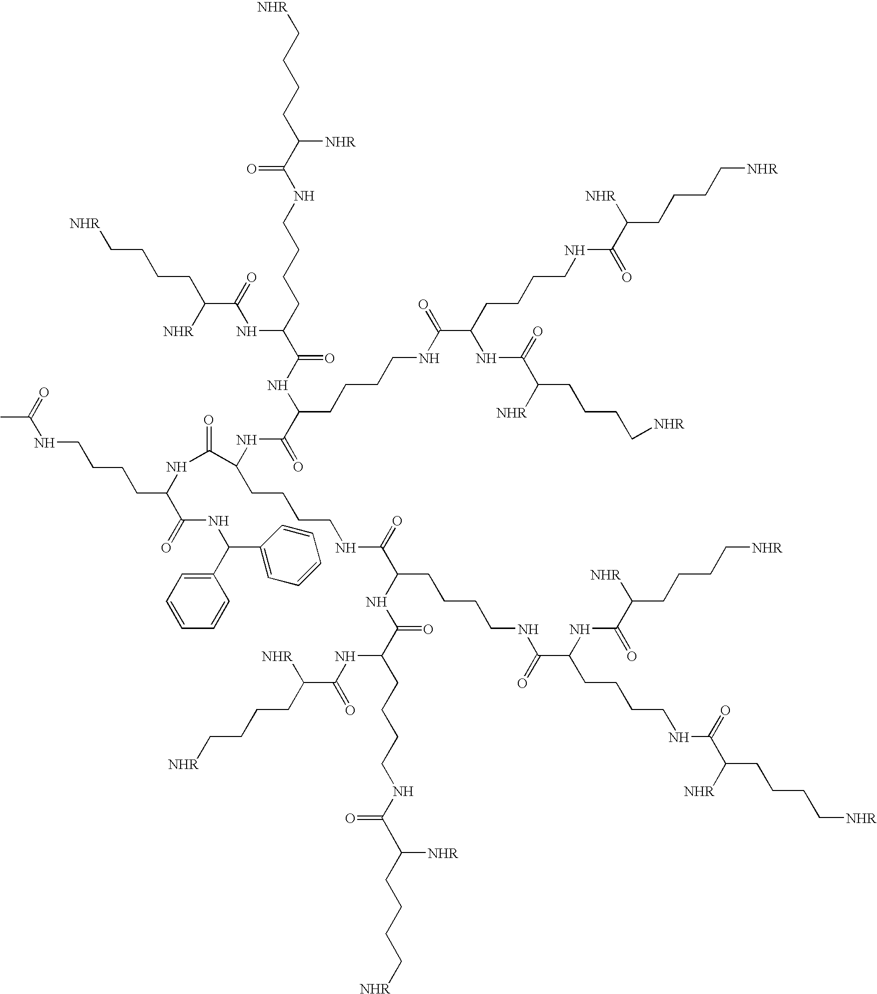 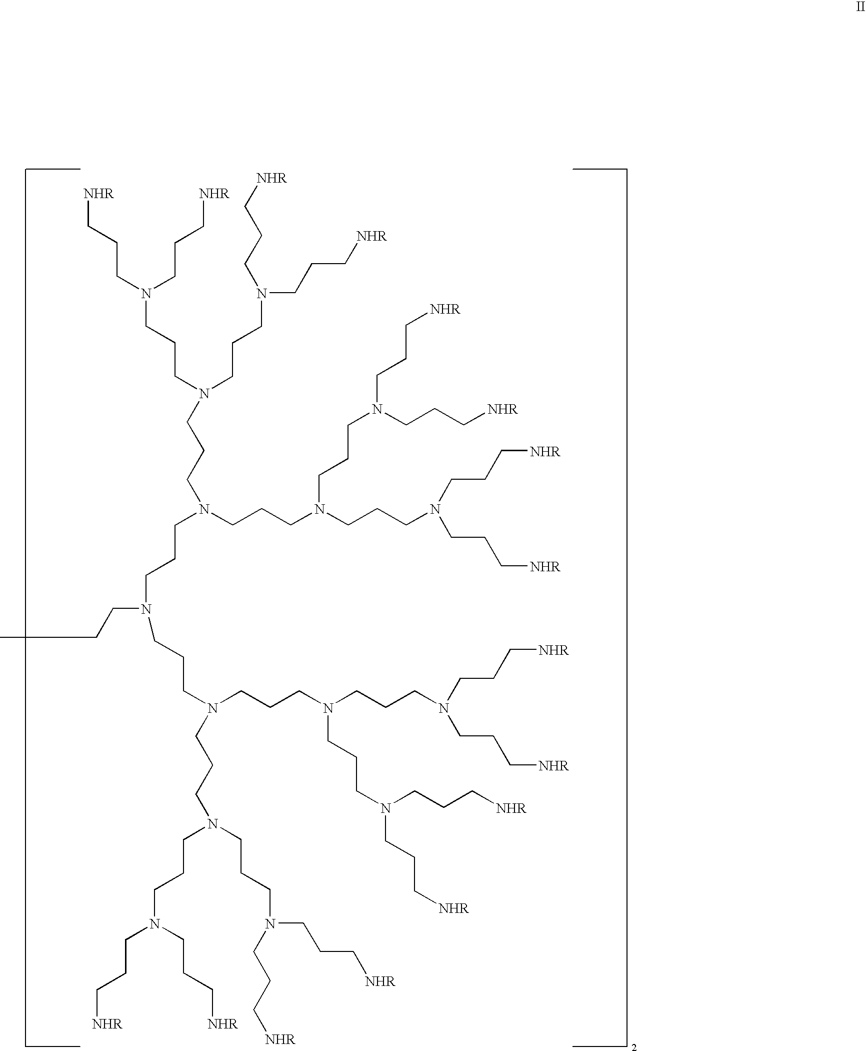 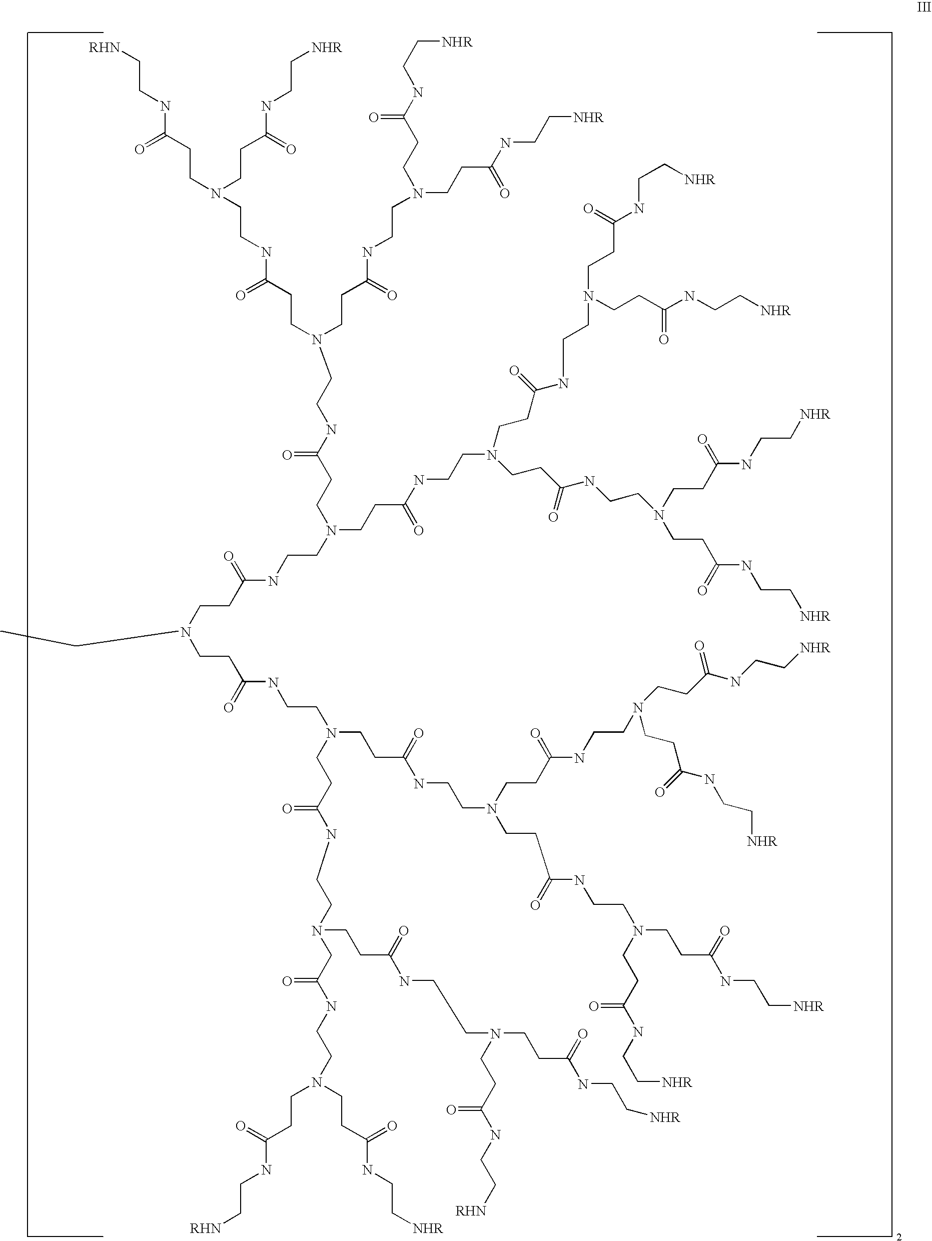
where R represents a group of the Formula (IV): 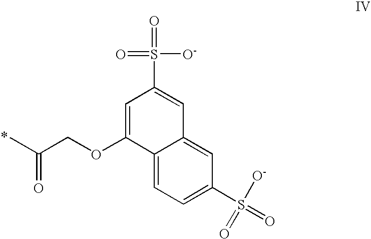
or a pharmaceutically acceptable salt thereof.

19. The composition according to claim 18 wherein the dendrimer molecule is the disodium salt of Formula (I).

20. The composition according to claim 17 in the form of a gel or film, optionally further comprising a pharmaceutically acceptable carrier.
